(19) United States
(12) Patent Application Publication
(10) Pub. No.: US 2013/0177875 A1
(43) Pub. Date: Jul. 11, 2013

(54) SYSTEMS AND METHODS FOR RECORDING DENTAL-IMPLANT INFORMATION

(71) Applicant: Chandur Wadhwani, Bellevue, WA (US)

(72) Inventor: Chandur Wadhwani, Bellevue, WA (US)

(21) Appl. No.: 13/737,454

(22) Filed: Jan. 9, 2013

Related U.S. Application Data
(60) Provisional application No. 61/584,973, filed on Jan. 10, 2012.

(51) Int. Cl. A61C 8/00; A61C 9/00; A61B 6/14

(52) U.S. Cl. CPC A61C 8/0089; A61C 8/0022; A61B 6/145; A61C 8/0001; A61C 9/00
USPC 433/174

(57) ABSTRACT

Alignment systems and methods for recording the orientation information of a dental implant are disclosed. The alignment system is a plate-type positioning device used for recording and identifying the location of dental implants, standardizing the implant assessment and providing a means to identify the abutment screw position. The plate has a guide hole to position and locate the implant position relative to the plane of the plate. In one aspect, an alignment system includes a plate with a hole to receive a positioning rod. The rod has a tip that engages the screw head of a dental implant, aligning the rod with the long axis of the dental implant and placing the plane of the plate at approximately 90 degrees to the long axis.

### CROSS-REFERENCE TO RELATED APPLICATION

This application claims the benefit of Provisional Application No. 61/584,973, filed Jan. 10, 2012.

### TECHNICAL FIELD

Alignment systems and methods for recording the long axis of a dental implant are disclosed, and, in particular, systems and methods for aligning an x-ray beam with a dental implant for acquiring multiple images of the implant over time are disclosed.

### BACKGROUND

Exact knowledge of a dental implant location and relation to other dental structures for monitoring bone levels is essential. The ability to make successive radiographs and compare these to each other is necessary to assess the health of the bone surrounding the implant. Frequently marginal or crestal bone levels are used to assess the health and successfulness of a dental implant. However, the radiographs must be captured with consistent orientation with respect to the position of the implant in order to accurately assess the health of the bone. FIG. 1 shows a series of three radiographs that demonstrate problems with radiograph alignment in assessing the health of surrounding bone. The three radiographs show three different views of a dental implant 101, but because each radiograph was captured from a different angle it is impossible to determine whether bone levels surrounding the implant 101 have actually changed or whether apparent discrepancies between radiographs represent a superimposition of bone from different angles. In order to achieve consistent radiographs, the angle between the long axis of the implant to the central axis of the x-ray beam and film position must be controlled. A 90-degree angle of incidence of the x-ray beam to the long axis of an implant is ideal. This is known as the “paralleling technique,” which is considered the standard when making dental x-rays.

Mechanical connections of implant component parts also must be monitored over time in order to determine if the implant is functing as intended. Implant connections must be routinely checked and confirmed correct, but implant connections cannot be directly accessed. Radiography is also frequently used to evaluate implant connections. However, finding gaps, or assessing seating of components, is highly dependent upon the relative angle of the x-ray beam used to illuminate the implant. FIG. 2 shows four radiographs that demonstrate how different x-ray beam angles of incidence make it difficult to examine implant connections. The radiographs were captured for four different angles of an x-ray beam with respect to the long axis of a dental implant. Radiograph 201 is of the long axis of the implant located at approximately 90 degrees with respect to the central axis of the x-ray beam. Radiographs 202-204 are of the same implant rotated through 10, 20, and 30 degrees, respectively, away from 90 degrees. Note that for radiographs 202-204 it is not possible to assess implant connections, and for radiographs 201 and 202 it is almost impossible to determine whether the implant has been rotated at all. Only radiograph 201 can be used to assess connections of the component parts.

In recent years, relator devices have been developed to relate an implant long axis to the central axis of an x-ray beam. These devices typically require direct access to the screw head of the implant. However, once a dental restoration is attached to an implant, e.g. cemented to the implant, the screw head and thread become inaccessible without destroying a significant portion of the implant, limiting the ability to use a relator device again. FIGS. 3 show a screw head 301 of an implant 302 and a restoration 304 cemented to the screw head 301. Once, the restoration 304 has been cemented to the screw head 301, the ability to find the screw head 301 without destroying a large amount of the restoration 304 is often impossible. Frequently, the screw head can only be guessed at with no easy way of determining the location of the screw head beneath the restoration.

The orientation of an implant should be recorded so that, should the implant ever need to be disassembled for maintenance, hygiene, screw retrieval, or repair, the exact site of the screw head is known. In most instances, especially when the implant is angled or not central, it is impossible to locate the screw head accurately after the restoration is placed.

### BRIEF DESCRIPTION OF THE DRAWINGS

FIG. 1 shows a series of radiographs illustrating problems with image alignment.

FIG. 2 shows how angulation affects the ability to assess dental implant connections.

FIGS. 3 show a screw head of an implant and complete restoration placed over the screw head.

FIG. 4 shows an exploded isometric view of an example alignment system.

FIGS. 5A-5C show three additional views of the plate.

FIG. 6 shows an isometric view of an example plate.

FIG. 7 shows a perspective view of a plate attached to the bite block of a dental x-ray film holder.

FIG. 8 shows a side-elevation view of a positioning rod inserted into a guide hole of a plate.

FIGS. 9A-9E show an example implementation of an alignment system.

FIG. 10 shows a flow diagram of a method for using an alignment system to construct a dental restoration.

FIG. 11 shows a flow diagram of a method for using an alignment system to place a dental restoration.

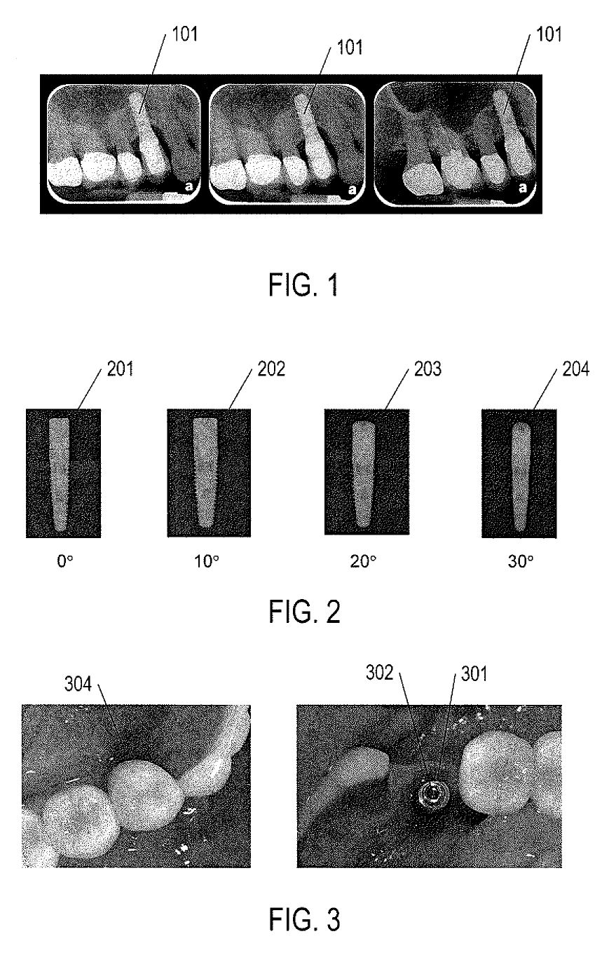

### DETAILED DESCRIPTION

Alignment systems and methods for recording the orientation information of a dental implant are disclosed. The alignment systems and methods enable alignment of the implant, x-ray beam and film to be reproduced. In particular, the alignment systems enable precise alignment of the long axis of a dental implant with the central beam axis of an x-ray beam to within about ±5° of perpendicular to the long axis of the dental implant. Alignment systems described herein have the advantage of being useful with or without a restoration in place. Although the alignment systems utilizes the screw head of a dental implant initially, at fabrication, additional reference points are produced that make the screw head reference redundant. As a result, a crown or other restoration can be placed and the reference position still maintained. The alignment systems align the implant long axis to a fixed reference point, allowing for precise location of the screw head and the long axis of the implant after a restoration has been placed. A guide hole in the alignment system may be used to mark the location of the screw head beneath the restoration and gain access to the screw head with minimal destruction of the restoration.

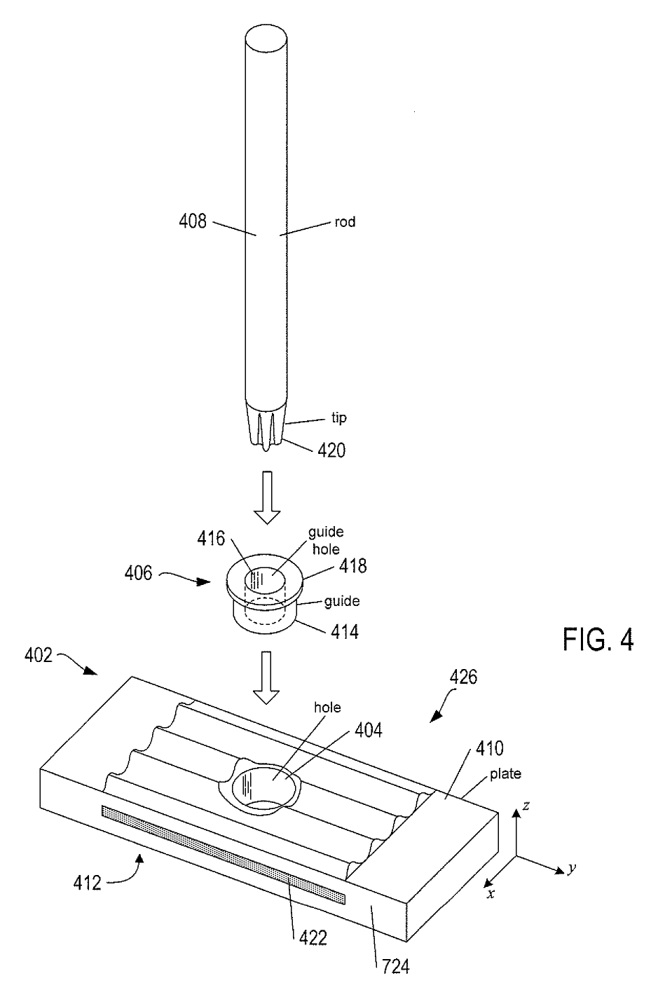

FIG. 4 shows an exploded isometric view of an example alignment system 400. The system 400 includes a plate 402 with a hole 404, a guide 406, and a positioning rod 408. The hole 404 spans the distance between an upper surface 410 and a lower surface 412 of the plate 402. The guide 406 includes a shaft 414 with an outer diameter that is slightly smaller than the diameter of the hole 404 to fit snuggly within the hole 404 and has an inner guide hole 416 with a diameter slightly smaller than the diameter of the rod 408. In the example of FIG. 4, the guide 406 includes a lip 418 to prevent the guide 406 from sliding out of the hole 404 when the rod 408 is inserted into the guide hole 416. The positioning rod 408 is a cylindrical shaft with a screw tip 420 located at one end. The screw tip 420 is shaped to fit within the contoured shape of a screw head of a dental implant. In other words, each dental implant has a screw with an exposed screw head that sits above the gum line. The screw head is contoured with a patterned impression or shape and the tip of the positioning rod is contoured with the same configuration as the screw tip used to engage and tighten the screw. The plate 402 includes a reference marker 422 located on the front edge 424 of the plate 402. The reference marker 422 can be a series of points or a wire-type radiopaque device that lies substantially parallel with the spatial or xy-plane of the plate 402. The reference marker 422 can be used as a reference on the radiographs for measurement purposes and can also be used to superimpose a series of radiographs to facilitate comparison of a series of radiographs for changes in bone density (e.g., subtraction radiography). The upper surface 410 also includes an embossed region 426 that is patterned to engage an embossed patterned of a bite block of a dental x-ray film holder such that when the embossed region engages the embossed pattern of a bit block, the plane of the plate 402 and the bite block are parallel, and the plane of the plate is substantially perpendicular to the long axis of the dental implant. It should be noted that the plate 402 can be fabricated from provisional or custom tray material, such as TruTray or Dentsply. The guide hole 416 diameter can range from about 1.9 millimeters to about 2.3 millimeters, depending on the diameter of the rod 408. For example, the guide hole 416 can have a diameter of about 2.1 millimeters.

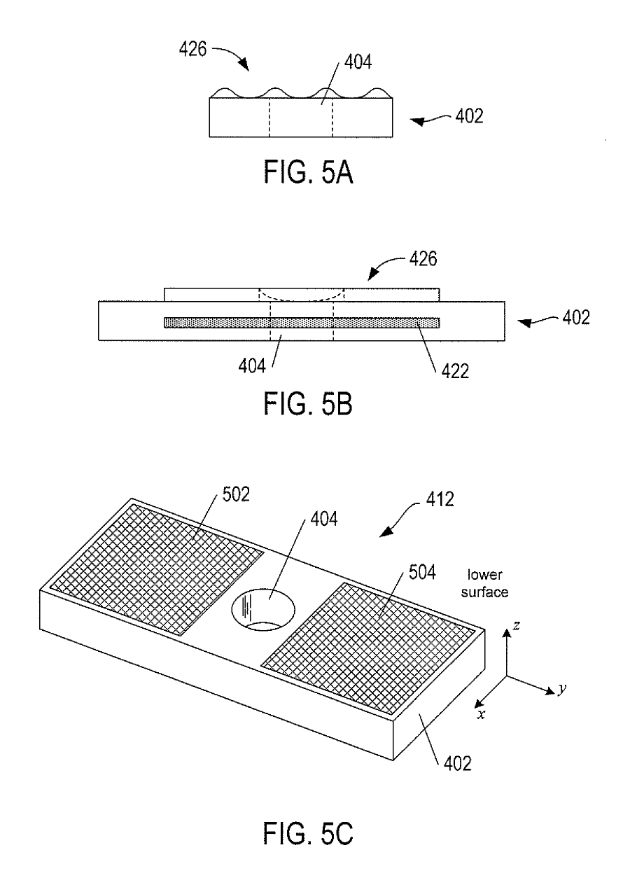

FIGS. 5A-5D show three additional views of the plate 402. FIG. 5A shows an end view of the plate 402, which reveals the smooth, wave-like raised surface of the embossed region 426. FIG. 5B shows a front view of the plate 402. FIGS. 5A and 5B both reveal the shape of the hole 404. FIG. 5C shows an isometric view of the lower surface 412 of the plate 402. The lower surface includes textured regions 502 and 504 composed of grooves, dips, hollows or recesses. The textured regions 502 and 504 act as retainers for adhesives and impression material to attach to the lower surface of the plate 402 to one or two teeth located adjacent to the implant.

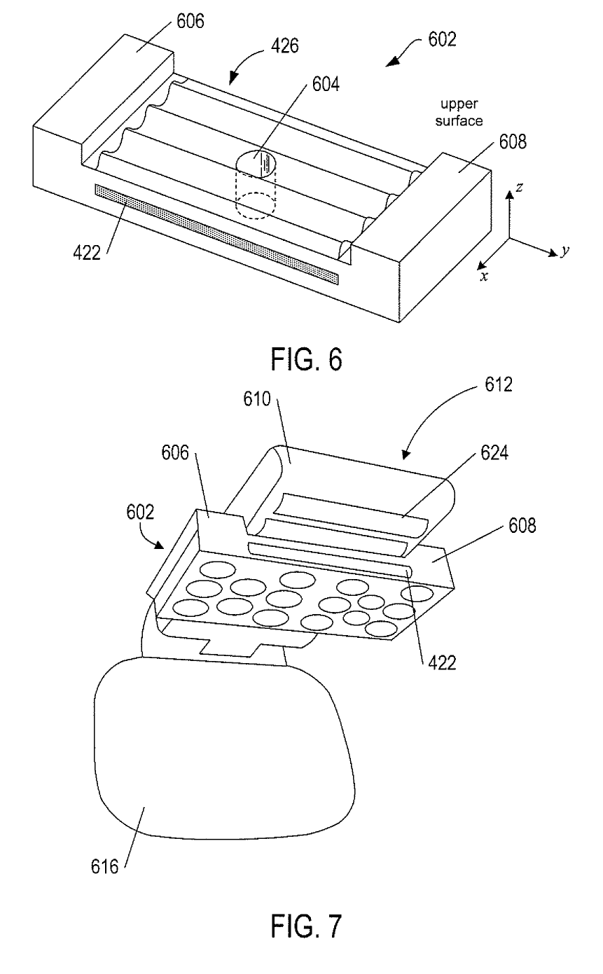

In other embodiments, the guide 406 can be omitted and/or the plate can include raised end surfaces that clamp the plate to the outside edges of a bite block of a dental x-ray film holder. FIG. 6 shows an isometric view of an example plate 602. The plate 602 is similar to the plate 402 in that the plate 602 includes an embossed region 426 to engage the embossed pattern of a bite block of a dental x-ray film holder. The plate 602 includes a hole 604 that serves as a guide hole with a diameter that is slightly smaller than the diameter of the rod 408. In this example, the plate 602 also includes raised end surfaces 606 and 608. The gap between the end surfaces 606 and 608 is approximately the same width or slightly smaller than the width of a bite block of a dental x-ray film holder. FIG. 7 shows a perspective view of the plate 602 attached to the bite block 610 of a dental x-ray film holder 612. The holder 612 includes a panel 616 for attaching x-ray film located approximately perpendicular to the plane of the bite block 610. As shown in FIG. 6B, raised surfaces 606 and 608 form a clamp that holds the plate 602 against the bite block 610 such that the embossed region (not shown) of the plate 602 engages the embossed pattern 614 of the bite block 610. As a result, when the plate 602 is attached to the bite block 612 of the holder 612, the plane of the plate 602 is located approximately perpendicular to the x-ray film attached to the panel 616.

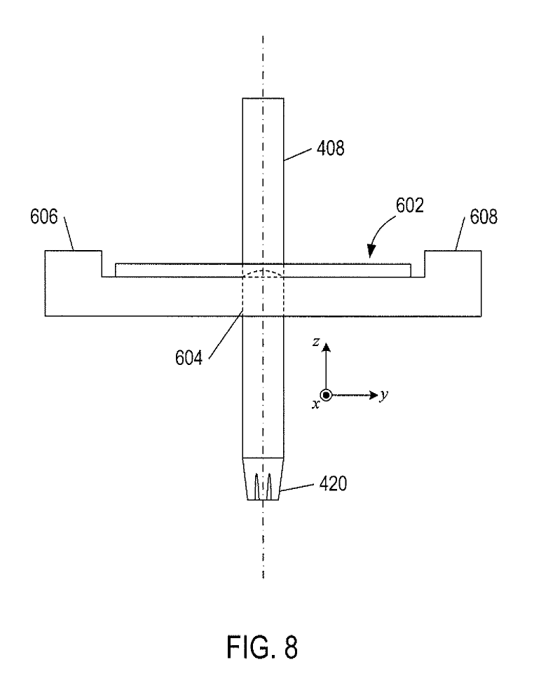

Returning to FIG. 4, when the positioning rod 408 is inserted into the guide hole 416 of the guide 406, which in turn is located within the hole 404 of the plate 402, the rod 408 is located perpendicular to the xy-plane of the plate 402. Likewise, when the positioning rod 408 is inserted into the guide hole 604 of the plate 602, the rod is located perpendicular to the xy-plane of the plate 602 as shown in the side-elevation view of FIG. 8.

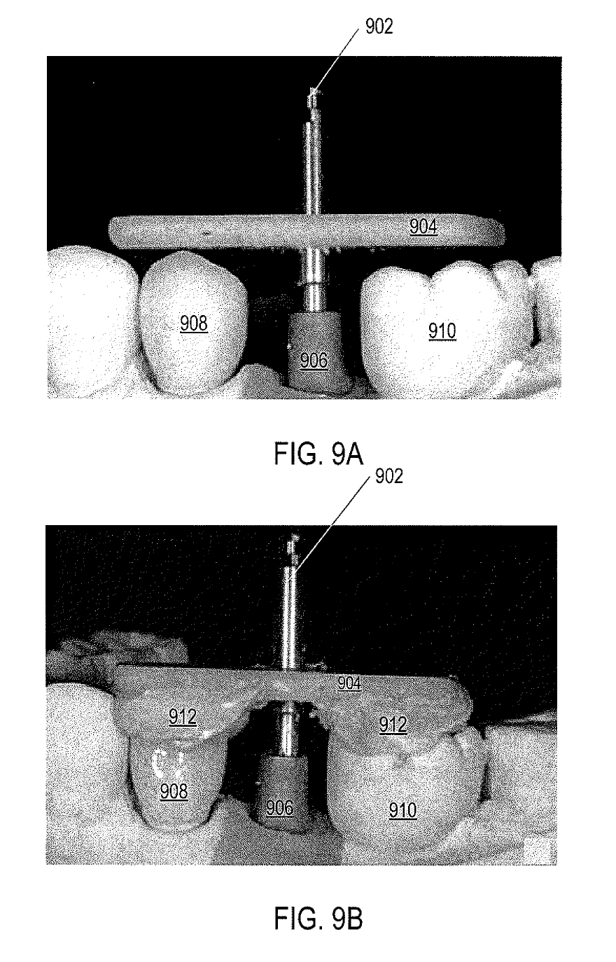

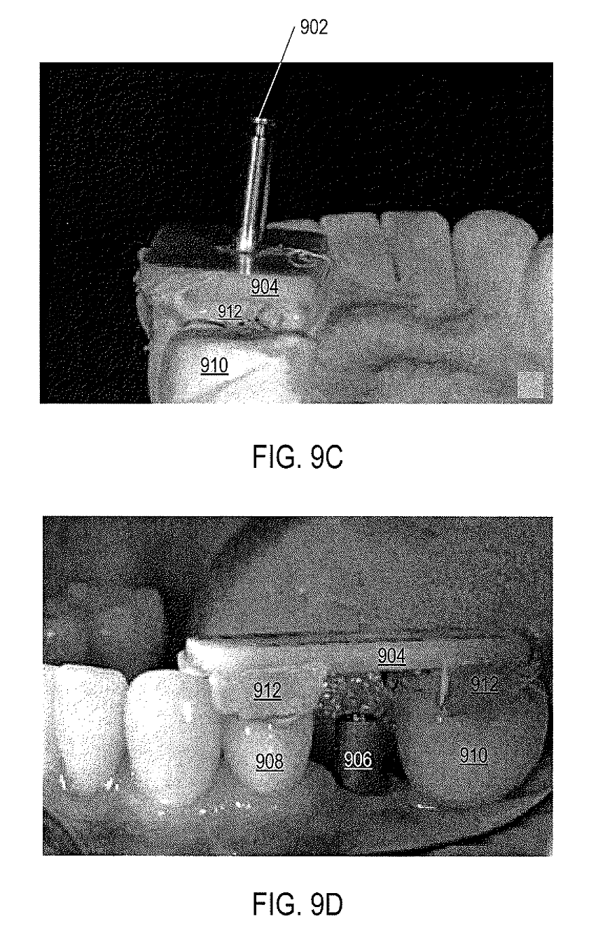

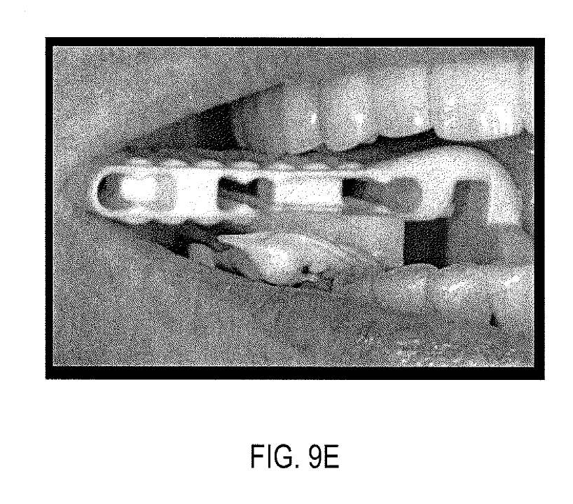

FIGS. 9A-9E show an example implementation of an alignment system. FIG. 9A shows a side-elevation view of a positioning rod 902 inserted into a guide hole of a plate 904. The rod tip (not shown) is inserted into the screw head 906 of a dental implant located beneath the gum line. The rod 902 is aligned with the long axis of the implant, and the guide hole in the plate 904 ensures that the plane of the plate 904 is located at approximately 90 degrees to the log axis of the implant. The plate 904 can slide up and down the rod 902 so that the plate may make contact with adjacent teeth 908 and 910, which relates the adjacent teeth 908 and 910 to the implant itself. As described above with reference to FIG. 5, the lower surface of the plate 904 has grooves, dips, hollows or holes that act as retainers for impression material to attach to the plate 904 to adjacent teeth 908 and 910. FIG. 9B shows a side-elevation view of the alignment system with impression material 912 disposed between the lower surface of the plate 904 and the adjacent teeth 908 and 910. The impression material 912 further locates and stabilizes the plate 904 relative to the implant such that the plane of plate 904 is located at approximately 90 degrees to the long axis of the implant and rod 902 is aligned with the long axis of the implant. FIG. 9C shows another view of the plate 904 attached to adjacent teeth 908 and 910 with impression material 912. FIG. 9D shows the positioning rod removed such that the guide hole in the plate is located directly above the screw head 906. The plate 904 relates the implant long axis to adjacent sites and the guide hole can be used to relocate the implant screw following placement of a crown or restoration over the screw head 906. As a result, the crown or restoration need not be removed to determine the long axis of the implant. The plate 904 and impression material 912 record the site of the screw head and the long axis of the implant. The plate 904 with impression material 912 attached can be removed, stored and repositioned on the adjacent teeth 908 and 910 after the restoration is complete to determine the location of the screw head 906 and the long axis of the implant. As described above, the embossed region of the upper surface of the plate 904 engages a bite block of an x-ray film holder such that the plate 904 and the bite block lie in the same plane, which is substantially perpendicular to the x-ray film attached to the panel of the holder, as described above with reference to FIG. 7. As a result, successive radiographs can be made and compared to each other for health and monitoring purposes. Also the x-ray tube angle guidance attached to the x-ray film holder is transferred directly to the plate because it lies in the same special plane, and is held in position by engagement between the bit block and the plate 904 as shown in FIG. 9E. This alignment can be used for assessing the quality of dental implant connections.

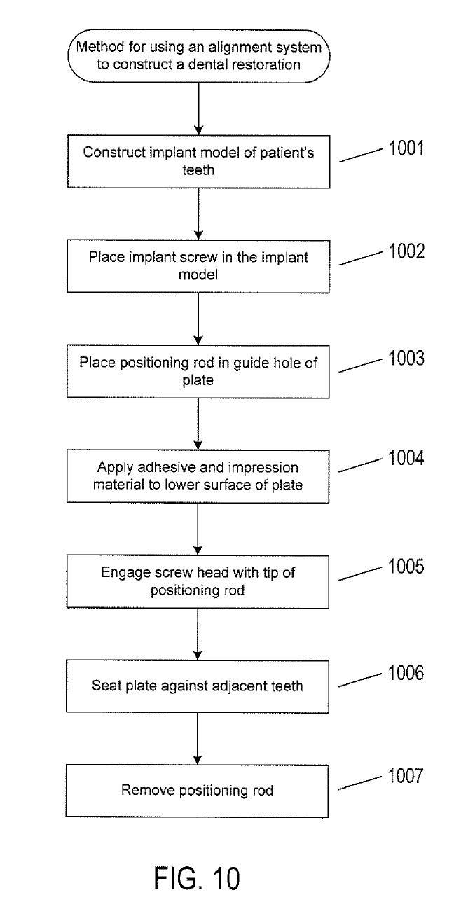

The alignment system can be used during construction of a dental restoration in a lab. FIG. 10 shows a flow diagram of a method for using an alignment system to construct a dental restoration. In block 1001, an implant model of a patient's teeth is constructed. In block 1002, the screw of a dental implant is placed in the implant model. In block 1003, a positioning rod is placed through the guide hole of a plate. In block 1004, adhesive is placed on the lower surface of the plate and impression material is placed on the adhesive. In block 1005, the plate is seated over the teeth adjacent to the implant, as shown in FIGS. 9B and 9C, and the screw head is engaged by the tip of the positioning rod as it is pushed and gently rotated through the guide hole in the plate to locate the long axis of the screw so that the plane of the plate is located at approximately 90 degrees (i.e., perpendicular) to the long axis of the implant. In block 1006, the plate is simultaneously gently seated against the adjacent teeth sites and the impression material is allowed to set. In block 1007, the positioning rod is removed which has set on the model.

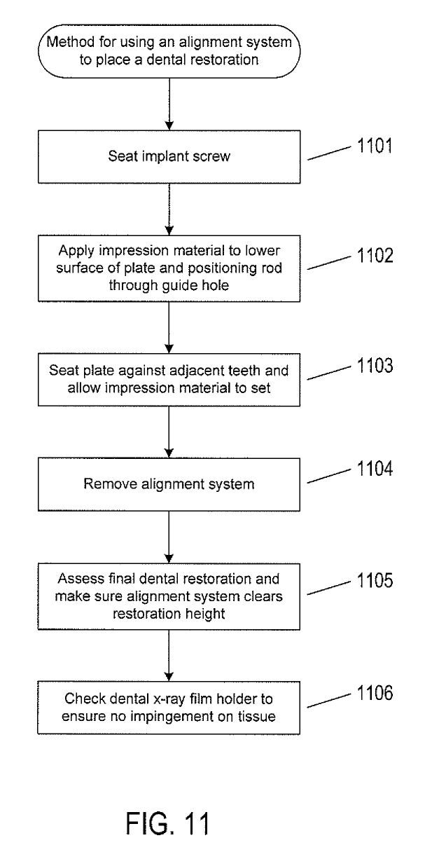

The alignment system can be used during placement of a dental restoration. FIG. 11 shows a flow diagram of a method for using an alignment system to place a dental restoration. In block 1101, the implant screw is seated which can also be done with the abutment in place. In block 1102, the lower surface of the plate is loaded with impression material and the positioning rod is placed through the guide hole to engage the screw head and locate the long axis of the implant. In block 1103, the plate is seated against adjacent teeth and the impression material is allowed to set. In block 1104, the alignment system with implant material attached to the lower surface of the plate is removed, checked to confirm that no implant material is over the implant site. In block 1105, the final dental restoration is tried and the alignment system is checked to make sure that it clears the crown height. If not, blocks 1101-1104 are repeated with more volume of impression material to raise the plate above the teeth. In block 1106, the dental x-ray film holder is checked, as shown in FIG. 9E, to confirm that it does not impinge on the tissue. If it does, then the plate may be raised above the teeth before completing the device. The plate with impression material attached to the lower surface of the plate now contains all the subsequently needed and useful information for locating the screw head and orientation of the long axis of the implant. In particular, the alignment system records information for angulation of x-rays, the upper surface of the plate is compatible with Rinn type devices that seat directly onto the alignment system. When the plate with impression material is used again, the plate will be approximately perpendicular to the long axis of the implant with the hole in the plate located above the screw head. As a result, the Rinn bite plane is now approximately perpendicular to the long axis of the implant. Sequential radiographs can be made and the implant followed very accurately. Also, during the restoration phase, the alignment system aids in determining if the fit of the components are accurate.

It should be noted that the guide hole in the plate allows for direct access to the screw's long axis. When the restoration needs to be removed, or the screw accessed, the guide hole indicates the location of the screw and guides the precise positioning of a dental drill bit above the screw. The alignment system can affirm the authenticity of the components being used by being supplied with the restoration from the lab. The alignment system can be used to record information such as the size of the dental implant and type of implant by having size and type indications recorded on the upper surface of the plate.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purposes of illustration and description. They are not intended to be exhaustive of or to limit the invention to the precise forms disclosed. Obviously, many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A dental implant alignment system comprising:
a plate with a first surface and an opposing, substantially parallel second surface and having a hole that extends from the first surface to the second surface; and
a positioning rod having a tip to engage a screw head of a dental implant, wherein the plate is approximately perpendicular to a long axis of the dental implant when the rod is inserted into the hole and the tip engages the screw head.

2. The system of claim 1, wherein the first surface comprises an embossed region to engage an embossed pattern of a bite block of a dental x-ray film holder.

3. The system of claim 1, wherein the second surface comprises at least one textured region to receive impression material and adhere the plate to one or more teeth located adjacent to the dental implant.

4. The system of claim 1, wherein the hole is a guide hole with a diameter slightly larger than the diameter of the positioning rod.

5. The system of claim 1 further comprising a guide to be inserted into the hole, the guide including a guide hole with a diameter slightly larger than the diameter of the positioning rod.

6. The system of claim 1 further comprises an x-ray reference marker located on an edge of the plate.

7. The system of claim 1, wherein the rod to be located at about 90 degrees with respect to the plane of the plate when the rod is inserted into the hole.

8. The system of claim 1, wherein the rod to be substantially aligned with the long axis of the dental implant when the tip is inserted into the screw head.

9. The system of claim 1, wherein the plate further comprises two raised regions to clamp the plate against a bite block of a dental x-ray film holder.

10. A method for recording screw head and long axis information of a dental implant, the method comprising:
placing a positioning rod with a tip in a guide hole of a plate, wherein the tip is contoured to engage the screw head;
applying impression material to a lower surface of the plate;
engaging the tip with the screw head such that the rod is substantially aligned with the long axis of the dental implant;
seating the plate such that the impression material lies between the lower surface and one or two teeth located adjacent to the implant such that the long axis of the dental implant is located approximately perpendicular to the plate and the hole is located above the screw head; and
removing the rod and plate with impression material attached, the plate with attached impression material records the location of the screw head and orientation of the implant.

11. The method of claim 10, wherein the plate further comprises an upper surface located opposite the lower surface, the upper surface comprises an embossed region to engage an embossed pattern of a bite block of a dental x-ray film holder.

12. The method of claim 10, wherein the hole is a guide hole with a diameter slightly larger than the diameter of the positioning rod.

13. The method of claim 10 further comprises inserting a guide into the hole, the guide including a guide hole with a diameter slightly larger than the diameter of the positioning rod.

14. The method of claim 10, wherein the plate further comprises a reference marker located on an edge of the plate.

15. The method of claim 10, wherein the rod to be located at about 90 degrees with respect to the plane of the plate when the rod is placed into the hole.

16. The method of claim 10, wherein the plate further comprises two raised regions to clamp the plate against a bite block of a dental x-ray film holder.
